(19) United States
(12) Patent Application Publication
(10) Pub. No.: US 2009/0117098 A1
(43) Pub. Date: May 7, 2009

(54) Complement C1Q Inhibitors For The Prevention And Treatment Of Glaucoma

(75) Inventors: Allan R. SHEPARD, Fort Worth, TX (US); Abbot F. Clark, Arlington, TX (US); Peter G. Klimko, Fort Worth, TX (US); Martin B. Wax, Westlake, TX (US)

Correspondence Address: ALCON, IP LEGAL, TB4-8, 6201 SOUTH FREEWAY, FORT WORTH, TX, 76134, US

(73) Assignee: ALCON RESEARCH, LTD., Fort Worth, TX (US)

(21) Appl. No.: 12/262,841

(22) Filed: Oct. 31, 2008

Related U.S. Application Data
(60) Provisional application No. 60/986,023, filed on Nov. 7, 2007.

(51) Int. Cl. A61K 39/395; A61P 27/06

(52) U.S. Cl. 424/130.1

(57) ABSTRACT

The invention concerns in one embodiment a method of treating glaucoma or elevated intraocular pressure comprising administering a pharmaceutically effective amount of a composition comprising a Complement C1q inhibitor. In another embodiment, the invention concerns a composition for the treatment of elevated intraocular pressure and glaucoma, the composition comprising a pharmaceutically effective amount of a Complement C1q inhibitor.

### CROSS-REFERENCE TO RELATED APPLICATION

This application claims priority under 35 U.S.C. §119 to U.S. Provisional Patent Application No. 60/986,023 filed Nov. 7, 2007, the entire contents of which are incorporated herein by reference.

### TECHNICAL FIELD OF THE INVENTION

The present invention is generally related to treatments for ocular hypertension and glaucoma, and more specifically related to inhibitors of complement C1q for the treatment of ocular hypertension and glaucoma.

### BACKGROUND OF THE INVENTION

The disease state referred to as glaucoma is characterized by a permanent loss of visual function due to irreversible damage to the optic nerve. The several morphologically or functionally distinct types of glaucoma are typically characterized by elevated intraocular pressure (IOP), which is considered to be causally related to the pathological course of the disease. Ocular hypertension is a condition wherein intraocular pressure is elevated, but no apparent loss of visual function has occurred; such patients are considered to be at high risk for the eventual development of the visual loss associated with glaucoma. If glaucoma or ocular hypertension is detected early and treated promptly, loss of visual function or the progressive deterioration thereof can often be ameliorated.

Drug therapies that have proven to be effective for the treatment of glaucoma and ocular hypertension include agents that reduce intraocular pressure. These agents comprise both agents that decrease aqueous humor production and agents that increase the outflow facility. Such therapies are in general administered by one of two possible routes, topically (direct application to the eye) or orally. However, pharmaceutical ocular anti-hypertension approaches have exhibited various undesirable side effects. For example, miotics such as pilocarpine can cause blurring of vision, headaches, and other negative visual side effects. Systemically administered carbonic anhydrase inhibitors can also cause nausea, dyspepsia, fatigue, and metabolic acidosis. Certain prostaglandins cause hyperemia, ocular itching, and darkening of eyelashes and periorbital skin. Such negative side-effects may lead to decreased patient compliance or to termination of therapy such that normal vision continues to deteriorate. Additionally, there are individuals who simply do not respond well when treated with certain existing glaucoma therapies. For example, some patients with glaucomatous field loss have relatively low intraocular pressure and may not respond to treatment using only agents that lower and/or control IOP. Further, many current therapies address only disturbances in intraocular pressure and may not resolve other problems associated with glaucoma, such as retinal ganglion cell (RGC) loss. There is, therefore, a need for other therapeutic agents for the treatment of glaucoma and ocular hypertension.

Inappropriate activation of the complement cascade has been suggested to occur in the pathogenesis of glaucoma (Stasi et al., IOVS 2006, Vol. 47:1024; Kuehn et al., Exp Eye Res 2006, Vol. 83:620). The complement system complements and amplifies the normal antibody response to foreign pathogens and consists of either the classical, MB-lectin, or alternative pathways. Certain members of the complement cascade are elevated in the rat model of ocular hypertension (Kuehn et al.) and in DBA/2J mice (Stasi et al.). In particular, complement component C1q was found to be elevated in the retinal ganglion cell (RGC) layer of the rats and mice. C1q was also detected in retinas of laser-induced hypertensive monkey eyes and some human glaucomatous eyes (Stasi et al.).

Complement C1q is a member of the classical complement pathway and is involved in the initial binding to antigen. Subsequent activation of proteases C1r, C1s, and C2-C4 results in amplification of the complement process and formation of the classical C3 convertase C4b C2b . . . C3 convertase is a central mediator of the complement cascade leading to formation of the Membrane Attack Complex (MAC). None of the above reports suggest the treatment of glaucoma using Complement C1q inhibitors.

### BRIEF SUMMARY OF THE INVENTION

The invention relates to the treatment of glaucoma and ocular hypertension using inhibitors of Complement C1q. In addition, embodiments of the present invention recognize that Complement C1q inhibitors may prevent and/or treat retinal ganglion cell (RGC) cell loss and other deleterious effects of ocular hypertension and glaucoma. Delivery of the inhibitors occurs via topical ocular, subtenons, subconjunctival, intracameral, juxtascleral, intravitreal, subretinal, or transcleral administration in preferred embodiments. Administration of the Complement C1q inhibitors allow the inhibitors to reach appropriate target tissue, such as the ocular layer containing retina ganglion cells (RGCs), at therapeutic levels thereby alleviating and preventing ocular damage resulting from glaucoma and providing neuroprotection.

Another embodiment of the present invention is a method of treating glaucoma or elevated intraocular pressure comprising administering a pharmaceutically effective amount of a composition comprising a Complement C1q inhibitor.

A non-comprehensive list of complement C1q inhibitors within the scope of the present invention include small molecule inhibitors; inhibitory antibodies; aptamers; non-antibody proteins that bind to and inactivate C1q; and agents that inhibit the expression of C1q, such as small interfering RNAs (siRNA), short hairpin RNAs (shRNA), ribozymes, deoxyribozymes, and antisense RNAs. The amount of complement C1q inhibitor present in the composition of the invention will typically be from 0.01% to 10% percent by weight.

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention when considered in connection with any accompanying figures. However, figures provided herein are intended to help illustrate the invention or assist with developing an understanding of the invention, and are not intended to be definitions of the invention's scope.

### BRIEF DESCRIPTION OF THE DRAWINGS

A more complete understanding of the present invention and the advantages thereof may be acquired by referring to the following description, taken in conjunction with the accompanying drawings in which like reference numbers indicate like features and wherein:

FIG. 1 provides an overview of the complement system, illustrating the classical, lectin, and alternative pathways.

### DETAILED DESCRIPTION OF THE INVENTION

### I. DEFINITIONS

Unless defined otherwise, technical and scientific terms used herein have the same meaning as commonly understood by one of ordinary skill in the art to which this invention belongs. For purposes of the present invention, the following terms are defined below.

The term “intraocular pressure” or “IOP” as used herein refers to the pressure of the fluid inside the eye. This pressure varies among individuals, for example, IOP may become elevated due to anatomical problems, inflammation of the eye, as a side-effect from medication or due to genetic factors. Elevated intraocular pressure is a significant risk factor for glaucoma.

The terms “prevention” and “preventing” as used herein are used according to their ordinary and plain meaning to mean “acting before”. In the context of a particular disease or health-related condition, those terms refer to administration or application of an agent, drug, or remedy to a subject or performance of a procedure or modality on a subject for the purpose of blocking the onset of a disease or health-related condition. For example, a subject that does not yet meet the clinical criteria or exhibit the symptoms of glaucoma, but does exhibit increases in intraocular pressure may be administered a composition of the present invention to prevent or delay the onset of glaucoma or perhaps reduce the severity of the condition.

The term “subject” as used herein refers to either a human or non-human, such as primates, mammals, and vertebrates. In particular embodiments, the subject is a human. Yet further, the terms “subject” and “patient” can be used interchangeably.

As used herein, the terms “therapeutically effective” or “treatment” refer to anything that promotes or enhances the well-being of the subject with respect to the medical treatment of his or her condition. This includes, but is not limited to, a reduction in the frequency or severity of the signs or symptoms of a disease.

### II. COMPLEMENT C1q INHIBITORS

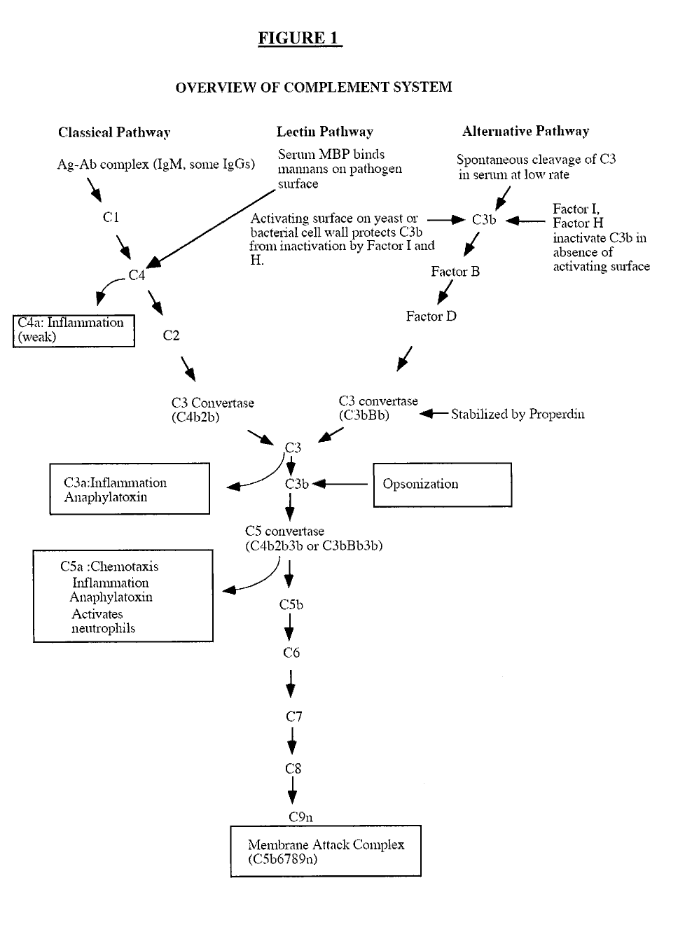

The complement system complements and amplifies the body's antibody response to foreign pathogens and is composed of three pathways: classical, MB-lectin, and alternative (FIG. 1). In the classical complement pathway, C1q is part of the C1 complex, which comprises a single C1q molecule bound to two molecules each of the zymogens (that is, inactive enzymes) C1r and C1s. C1q has six globular heads, linked together by a collagen-like tail, which surround the (C1r:C1s)₂ complex. Binding of more than one of the C1q heads to a pathogen surface causes a conformational change in the (C1r:C1s)₂ complex, which leads to activation of an autocatalytic enzymatic activity in C1r; the active form of C1r then cleaves its associated C1s to generate an active serine protease.

Once activated, the C1s enzyme acts on the next two components of the classical pathway, cleaving C4 and then C2 to generate two large fragments, C4b and C2b, which together form the C3 convertase C4b C2b of the classical pathway. In the first step, C1s cleaves C4 to produce C4b, which binds covalently to the surface of the pathogen. The covalently attached C4b then binds one molecule of C2, making it susceptible to cleavage by C1s. C1s cleaves C2 to produce the large fragment C2b, which is itself a serine protease. The C4b C2b C3 convertase complex which then forms on the surface of the pathogen is a serine protease that cleaves large numbers of C3 molecules to produce C3b molecules, which coat the pathogen surface. C3b is an opsonization agent that can ligate cognate receptors on phagocytic cells such as macrophages, leading to pathogen phagocytosis. Additionally, the C4bC2b C3 convertase can bind to a molecule of C3b to form the C5 convertase, C4b C2b C3b. The C5 convertase catalyzes the formation of the membrane attack complex (MAC) onto pathogen surfaces, leading to pathogen lysis and death.

Inhibition of C1q function by a variety of means, such as inhibition of its expression, binding by an anti-C1q antibody or aptamer, or inhibition of its serine protease activity, represents a strategy for reducing activation of the classical complement system, by reducing formation of the classical C3 convertase C4b C2b. This will reduce the contribution of inappropriate classical complement system activation to glaucoma pathology/tissue destruction via downstream events such as C3 convertase formation, cell phagocytosis, and cell lysis due to MAC formation. Inhibition of C1q as a means of preventing and/or ameliorating glaucoma-associated disease pathology in man has not been attempted.

As used herein, the term “Complement C1q inhibitor” refers to any molecule that is capable of decreasing, inhibiting, or downregulating the activity, expression or function of Complement C1q. Complement C1q inhibitors may include fragments or parts of naturally-occurring compounds or may be found as active combinations of known compounds which are otherwise inactive. The inhibitors can be nucleic acids, polypeptides, small molecules, antibodies, etc. It is proposed that compounds isolated from natural sources, such as animals, bacteria, fungi, plant sources, including leaves and bark, and marine samples may be assayed as candidates for the presence of potentially useful Complement C1q inhibitors. It will be understood that the pharmaceutical agents to be screened could also be derived or synthesized from chemical compositions or man-made compounds.

In certain embodiments, a small molecule library that is created by chemical genetics may be screened to identify a candidate substance that may be a Complement C1q inhibitor of the present invention. Using this method, one may simply acquire, from various commercial sources, small molecule libraries that are believed to meet the basic criteria for useful drugs. Screening of such libraries, including combinatorially generated libraries, is a rapid and efficient way to screen a large number of related (and unrelated) compounds for activity. Combinatorial approaches also lend themselves to rapid evolution of potential drugs by the creation of second, third and fourth generation compounds modeled of active, but otherwise undesirable compounds. It will be understood that an undesirable compound includes compounds that are typically toxic, but have been modified to reduce the toxicity or compounds that typically have little effect with minimal toxicity and are used in combination with another compound to produce the desired effect.

It is recognized that compounds disclosed herein can contain one or more chiral centers. This invention contemplates all enantiomers, diastereomers, and mixtures of compounds disclosed herein. Furthermore, certain embodiments of the present invention comprise pharmaceutically acceptable salts of disclosed compounds. Pharmaceutically acceptable salts comprise, but are not limited to, soluble or dispersible forms of compounds that are suitable for treatment of disease without undue undesirable effects such as allergic reactions or toxicity. Representative pharmaceutically acceptable salts include, but are not limited to, acid addition salts such as acetate, citrate, benzoate, lactate, or phosphate and basic addition salts such as lithium, sodium, potassium, or aluminum.

### III. MODES OF DELIVERY

The Complement C1q inhibitory compounds of the present invention can be incorporated into various types of ophthalmic formulations for delivery. The compounds may be delivered directly to the eye (for example: topical ocular drops or ointments; slow release devices such as pharmaceutical drug delivery sponges implanted in the cul-de-sac or implanted adjacent to the sclera or within the eye; periocular, conjunctival, sub-tenons, intracameral, intravitreal, juxtascleral or intracanalicular injections) using techniques well known by those of ordinary skill in the art. It is further contemplated that the Complement C1q inhibitory compounds of the invention may be formulated in intraocular inserts or implantable devices.

### IV. FORMULATIONS

The Complement C1q inhibitory compounds disclosed herein are preferably incorporated into topical ophthalmic formulations for delivery to the eye. The compounds may be combined with ophthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving a compound in a physiologically acceptable isotonic aqueous buffer. Further, the ophthalmic solution may include an ophthalmologically acceptable surfactant to assist in dissolving the compound. Furthermore, the ophthalmic solution may contain an agent to increase viscosity such as hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient is combined with a preservative in an appropriate vehicle such as mineral oil, liquid lanolin, or white petrolatum. Sterile ophthalmic gel formulations may be prepared by suspending the compound in a hydrophilic base prepared from the combination of, for example, carbopol-974, or the like, according to the published formulations for analogous ophthalmic preparations; preservatives and tonicity agents can be incorporated.

Complement C1q inhibitory compounds are preferably formulated as topical ophthalmic suspensions or solutions, with a pH of about 4 to 8. The compounds are contained in the topical suspensions or solutions in amounts sufficient to treat patients experiencing elevated IOP and/or glaucoma. The compounds will normally be contained in these formulations in an amount 0.01 to 5 percent by weight/volume (“w/v %”), but preferably in an amount of 0.25 to 2 w/v %. For topical presentation 1 to 2 drops of these formulations would be delivered to the surface of the eye 1 to 4 times per day, according to the discretion of a skilled clinician.

The Complement C1q inhibitors can also be used in combination with other elevated IOP or glaucoma treatment agents, such as, but not limited to, rho kinase inhibitors, β-blockers, prostaglandin analogs, carbonic anhydrase inhibitors, α₂ agonists, miotics, and neuroprotectants.

### V. EXAMPLES

The following examples are provided to illustrate certain embodiments of the invention, but should not be construed as implying any limitations to the claims.

### EXAMPLE 1

This example illustrates the composition of a representative pharmaceutical formulation for intravitreal ophthalmic administration containing a Complement C1q inhibitor of the present invention. A similar fomulation could be used for a Complement C1q inhibitor of the present invention.

| Ingredient | Amount (w/v, %) |
|---|---|
| Complement C1q inhibitor | 0.1-10 |
| PEG 400 | 10 |
| Polysorbate 80 | 0.5 |
| HPMC 2910 | 0.5 |
| Dibasic sodium phosphate, dodecahydrate | 0.18 |
| Sodium hydroxide | q.s. to pH |
| Hydrochloric acid | q.s. to pH |
| Water for Injection | q.s. to 100% |

### EXAMPLE 2

This example illustrates the composition of a representative pharmaceutical formulation containing a Complement C1q inhibitor of the invention, for posterior juxtascleral, anterior juxtascleral, or periocular administration.

| Ingredients | Amount (w/v, %) |
|---|---|
| Complement C1q inhibitor | 5 |
| PEG 400 | 5 |
| Polysorbate 80 | 0.5 |
| HPMC 2910 | 0.5 |
| Dibasic sodium phosphate, dodecahydrate | 0.18 |
| Sodium Chloride | 0.17 |
| Sodium hydroxide | q.s. to pH |
| Hydrochloric acid | q.s. to pH |
| Water for Injection | q.s. to 100% |

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

### VI. REFERENCES

Furlong, S. T., Dutta, A. S., Coath, M. M., Gormley, J. J., Hubbs, S. J., Lloyd, D., Mauger, R. C., Strimpler, A. M., Sylvester, M. A., Scott, C. W., and Edwards, P. D. (2000). C3 activation is inhibited by analogs of compstatin but not by serine protease inhibitors or peptidyl alpha-ketoheterocycles. Immunopharmacology 48, 199-212.
Morikis, D., Assa-Munt, N., Sahu, A., and Lambris, J. D. (1998). Solution structure of Compstatin, a potent complement inhibitor. Protein Sci 7, 619-627.
Sahu, A., Rawal, N., and Pangburn, M. K. (1999). Inhibition of complement by covalent attachment of rosmarinic acid to activated C3b. Biochem Pharmacol 57, 1439-1446.

What is claimed is:

1. A method of treating glaucoma or elevated intraocular pressure comprising:
administering a therapeutically effective amount of a composition comprising a Complement C1q inhibitor.

2. The method of claim 1 wherein said composition further comprises a compound selected from the group consisting of:
ophthalmologically acceptable preservatives, surfactants, viscosity enhancers, penetration enhancers, gelling agents, hydrophobic bases, vehicles, buffers, sodium chloride, and water.

3. The method of claim 1, further comprising administering, either as part of said composition or as a separate administration, a compound selected from the group consisting of:
β-blockers, prostaglandin analogs, carbonic anhydrase inhibitors, α₂ agonists, miotics, neuroprotectants, and any combination thereof.

4. The method of claim 1 wherein said composition comprises from about 0.01 percent weight/volume to about 5 percent weight/volume of said inhibitor.

5. The method of claim 1 wherein said composition comprises from about 0.25 percent weight/volume to about 2 percent weight/volume of said inhibitor.

6. A composition for the treatment of elevated intraocular pressure and glaucoma comprising:
a pharmaceutically effective amount of a Complement C1q inhibitor.

7. The composition of claim 6, further comprising a compound selected from the group consisting of:
ophthalmologically acceptable preservatives, surfactants, viscosity enhancers, penetration enhancers, gelling agents, hydrophobic bases, vehicles, buffers, sodium chloride, and water.

8. The composition of claim 6 wherein said composition comprises from about 0.01 percent weight/volume to about 5 percent weight/volume of said inhibitor.

9. The composition of claim 6 wherein said composition comprises from about 0.25 percent weight/volume to about 2 percent weight/volume of said inhibitor.

10. The composition of claim 6 wherein said composition further comprises a compound selected from the group consisting of:
β-blockers, prostaglandin analogs, carbonic anhydrase inhibitors, α₂ agonists, miotics, neuroprotectants, rho kinase inhibitors, and any combination thereof.
